(19) United States
(12) Patent Application Publication
(10) Pub. No.: US 2010/0145164 A1
(43) Pub. Date: Jun. 10, 2010

(54) REMOTE HEALTH MONITORING METHOD AND SYSTEM

(76) Inventor: Steven Howell, Perthshire (GB)

Correspondence Address: HOVEY WILLIAMS LLP, 10801 Mastin Blvd., Suite 1000, Overland Park, KS, 66210, US

(21) Appl. No.: 12/432,353

(22) Filed: Apr. 29, 2009

(30) Foreign Application Priority Data
Dec. 5, 2008 (GB) GB0822237.4

(51) Int. Cl. A61B 5/00

(52) U.S. Cl. 600/301

(57) ABSTRACT

New systems and methods for remotely monitoring the activity, security, and health of an individual are provided. The monitoring system can be used to monitor both residence security and typical residential activity that is linked to the well being and health of an individual and can alert a third party when there is a digression from a predetermined set parameters. The monitoring system can incorporate a number of sensing elements including door opening and closing detection elements, passive infrared sensors, energy sensors for detecting the turning on and off of general household appliances, weighing scales, systolic blood pressure or pulse pressure measurement apparatus, bioelectrical impedance analysis measurement apparatus, breath analysis, and blood-based biological marker detection and quantification.

### CROSS-REFERENCE TO RELATED APPLICATIONS

This application claims the priority benefit of a foreign-filed application, Great Britain Patent Application No. GB0822237.4, entitled REMOTE HEALTH AND SECURITY MONITORING, filed Dec. 5, 2008, incorporated by reference herein in its entirety.

### BACKGROUND OF THE INVENTION

1. Field of the Invention

The present invention is broadly concerned with novel systems and methods for remotely monitoring the overall health and activity of an individual in their residence.

2. Description of the Prior Art

There are numerous remote health monitoring products and services available. For example, some systems monitor daily weight in order to aid at-home management of patients. However, these products are limited in that they only monitor an individual's health. In addition, there are home security systems that monitor intruder and unwanted entry into residential and commercial properties. Recently, home energy monitoring meters have and are being introduced into residential homes. These energy meters monitor energy usage within the residence. Each of these types of monitoring systems requires their own separate infrastructures in place in order to operate. In addition, the large scale cost of implementing health monitoring systems across the general population has meant that they have not reached mass market.

Obesity is linked to the incidence of Type 2 diabetes, hypertension and dyslipidaemia. Mild obesity involving a body mass index (BMI) of 30+, is less dangerous to health than morbid obesity (BMI 40+) or malignant obesity (BMI 50+). An individual who is 40% overweight is twice as likely to die prematurely as an average-weight person. This effect is seen after 10 to 30 years of being obese. The impact of excess body fat on mortality depends not only on the amount of excess fat (BMI) but also on its distribution (waist circumference, waist-to-hip ratio).

Relations between categories of body mass index (BMI), cardiovascular disease risk factors, and vascular disease end points have been examined prospectively in Framingham Heart Study participants aged 35 to 75 years, who were followed up to 44 years. The primary outcome was new cardiovascular disease, which included angina pectoris, myocardial infarction, coronary heart disease, or stroke. Analyses compared overweight (BMI [calculated as weight in kilograms divided by the square of height in meters], 25.0-29.9) and obese persons (BMI> or =30) to a reference group of normal-weight persons (BMI, 18.5-24.9). The age-adjusted RR (confidence interval [CI]) for cardiovascular disease was increased among those who were overweight (men: 1.21 [1.05-1.40]; women: 1.20 [1.03-1.41]) and the obese (men: 1.46 [1.20-1.77]; women: 1.64 [1.37-1.98]). High population attributable risks were related to excess weight (BMI> or =25) for the outcomes hypertension (26% men; 28% women), angina pectoris (26% men; 22% women), and coronary heart disease (23% men; 15% women). Conclusions stated that the overweight category was associated with increased relative and population attributable risk for hypertension and cardiovascular sequelae.

A study conducted at the University of Tübingen, Germany, involved 314 people aged 18 to 69 (average age of 45) and measured their total body fat, visceral fat (the fat around the abdomen and internal organs), and subcutaneous fat (fat under the skin) with magnetic resonance tomography. The participants also underwent an oral glucose tolerance test to measure their insulin resistance. Subjects assigned by weight into four groups: (1) normal weight, (2) overweight, (3) obese with insulin sensitivity (i.e. no resistance) and (4) obese with insulin resistance. In summary, overweight and obese individuals had more visceral and total body fat than the normal weight individuals. Obese individuals with insulin resistance had more fat in skeletal muscles and the liver than obese individuals who were insulin sensitive. The insulin-resistant individuals had thicker walls in their carotid arteries, which is an early indicator of narrowing of the arteries or atherosclerosis, a risk factor for heart disease. The obese insulin-sensitive individuals had the same level of insulin sensitivity and artery wall thickness as the normal weight group.

Bioelectrical Impedance Analysis (BIA) is a commonly used method for estimating body composition using an electrical impedance method to calculate an estimate of fat-free body mass and body fat calculated from the difference in body weight. Recent technological improvements have made BIA a more reliable and therefore more acceptable way of measuring body composition. This measurement is amenable to foot plate measurement.

Increased systolic blood pressure is strongly correlated with aging and leads to increased stiffness of large arteries, increased pulse pressure, and the incidence of cardiac and vascular disease. In contrast diastolic BP increases until about age 55 and then declines. Pulse pressure (systolic-diastolic BP difference) increases with age. Downstream clinical benefits of treatment of systolic hypertension include reductions in stroke, myocardial infarction, heart failure, kidney failure, and overall cardiovascular disease morbidity and mortality. Pulse pressure, although robust as a risk indicator, is considerably less straightforward to use clinically than systolic BP. Pulse pressure has not yet been validated as a surrogate end point for morbidity or mortality in a prospective randomized clinical trial. Measurement of systolic blood pressure is not amenable to hand/foot format. Existing measurements are all clinically validated using cuff measurement. Several lines of strong evidence support the initiative to measure systolic BP. The value of systolic BP in risk prediction is convincingly demonstrated in 12-year data from >316,000 men screened for the Multiple Risk Factor Intervention Trial (MRFIT). As demonstrated in this large cohort, coronary heart disease death rates were almost linearly related to systolic BP at all levels of blood pressure.

Thus, it is an object of the present invention to provide methods of remotely monitoring an individual's health and/or security and/or energy consumption. It is also an objective to provide monitoring systems for monitoring and evaluating an individual's health and/or security and/or energy consumption and notifying or alerting a third party when data collected by the monitoring system falls outside of defined parameters. In this way, a health care worker or care giver can remotely monitor the activity of a patient or family member and be alerted when the individual being monitored deviates from the defined parameters of security and/or health. Likewise, a computer programmed with an algorithm can automatically monitor the activity of an individual and automatically trigger an alarm notifying the individual or a third party when the activity deviates from the defined parameters.

### SUMMARY OF THE INVENTION

The present invention overcomes the problems in the prior art by providing an inventive remote health monitoring method and system for monitoring an individual's activity, and correlating that activity with a determination of the health and/or safety status of the individual based upon a predetermined set of parameters.

In one aspect, there is provided a method of remotely monitoring the security of a property or the health of an individual using a remote health monitoring system. The systems comprises at least one of a first sensing element that acquires data regarding the movement of the individual, and at least one of a second sensing element that acquires data regarding the health of the individual.

In another aspect of the invention, there is provided a remote monitoring system for monitoring the activity of an individual. The system comprises at least one of a first sensing element that acquires data regarding the movement of the individual, and at least one of a second sensing element that acquires data regarding the health of the individual.

In yet another aspect, there is provided a method of remotely monitoring the activity of an individual. The method comprises collecting a first set of data from a sensing element located in the residence of said individual. The sensing element is selected from group consisting of a passive infrared sensor, a magnetic door strip sensor, an energy sensor, a water flow sensor, a gas flow sensor, a weight scale, a bioelectrical impedance sensor, a blood pressure sensor, a pulse pressure sensor, a finger-stick blood test, a blood glucose concentration test, a test for measuring natriuretic peptides, a test for measuring viral load, a human body fluid test, and combinations thereof, said human body fluid test being capable of detecting cholesterol, HDL, LDL, or combinations thereof. The first set of data is analysed to develop an activity profile for the individual. The activity profile defines parameters of “normal” activity levels of the individual. A second set of data is collected from the sensing elements, and is compared to the parameters. If the second set of data deviates from the predetermined parameters, a third party or the individual can be alerted to intervene and/or correct the problem.

### BRIEF DESCRIPTION OF THE DRAWINGS

FIG. 1(A)-(B) illustrate one embodiment of the inventive remote health and security monitoring system; and

FIG. 2 is a flow chart illustrating the processing of the data collected by the remote health and security monitoring system.

### DETAILED DESCRIPTION OF THE INVENTION

In more detail, there is provided a monitoring system comprising sensors or sensing elements around the residence of an individual. These sensors acquire data that is utilized to create an activity profile of the individual. For example, in a house, motion can be monitored throughout the house. In addition, usage of key electrical appliances, for example a kettle, refrigerator, television, radio, hairdryer, iron, and/or electric cooker and/or the usage of utility services, can be measured using an energy sensor. In addition, sensing elements that can take measurements relating to an individual's health can also be monitored.

The sensors can be of a number of formats. Preferably, the sensors are selected from the group consisting of motion sensors, energy sensors that monitor energy usage, water sensors that monitor water usage, gas sensors that monitor gas usage, and medical sensors/devices that can be used to test for a particular aspect of an individual's health.

Suitable medical sensors for use in the inventive monitoring system are selected from the group consisting of bioelectric foot plate sensors (e.g., various weighing scales and body composition monitoring products available from Tanita Corporation of America, Inc., Arlington Heights, Ill.), blood pressure cuff sensors (e.g., the Omron M10 IT Blood Pressure Monitor available from Omron Healthcare, Inc. Bannockburn, Ill.), finger-stick blood sample sensors, qualitative and quantitative human body fluid tests for cholesterol, HDL, LDL and similar tests (e.g., tests performed on the Cholestech LDX analyser, Cholestech, Hayward, Calif.), tests for measuring blood glucose concentration (e.g., the One Touch Ultra blood glucose monitoring system from Lifescan Ltd, High Wycombe, Buckinghamshire, England), tests for measuring natriuretic peptides, tests for measuring viral load (e.g., the CD4 and CD8 tests available from Bayer Healthcare and Roche), and combinations thereof.

Suitable motion sensors for use in the inventive monitoring system are selected from the group consisting of passive infrared motion sensors (e.g., the PC Patrol®-Wireless Infrared Motion Detector from Optex Inc., Chino, Calif.), magnetic contact breakers on internal and/or external doors (e.g., the Wireless Converter Magnetic Contact 15101X from Maplin Electronics Ltd, Wath-upon-Dearne, Rotherham, South Yorkshire, England) and various security alarm systems (e.g., Concord Express and Concord Integrated from U.S. Alarm, Hayward, Calif., ), and combinations thereof.

Suitable energy (i.e., electrical) sensors are selected from the group consisting of sensors that monitor energy usage (e.g., the Owl Wireless Energy Monitor from OWL, Ashbourne, Co Meath, UK; the Efergy Elite from Efergy UK Ltd.; the Eco-eye mini from Modern Moulds & Tools Ltd., West Sussex, UK; the Wattson 01 from DIY Kyoto, London, England; and the Onzo Smart Energy Kit, Onzo Ltd, London, UK), electrical sensors that monitor current (e.g., Hawkeye® Current Monitoring from Veris Industries, Portland, Oreg.), electrical sensors that monitor voltage (e.g., the AKCP Voltage Monitor sensor from AKCP Co., Ltd, Jatujak, Bangkok, Thailand), electrical sensors that monitor capacitance (e.g., Capacitive Sensors (KAS) from RECHNER Industrie-Elektronik GmbH, Lampertheim, Germany), and combinations thereof.

Suitable water sensors are selected from the group consisting of water flow meters (e.g., the FM MAG 8000W from RShydro, Bromsgrove, UK), water volume sensors, sensors that can measure the weight of water, sensors that can measure the height of water in a vessel, and combinations thereof.

Suitable gas sensors are selected from the group consisting of sensors that monitor gas flow (e.g., various meters from icenta CONTROLS Ltd., Salisbury, UK), sensors that monitor gas pressure (e.g., the CirruS™ Atmospheric Pressure Gas Monitoring system from Cirrus Ltd, UK; and gas pressure monitors from Honeywell), and combinations thereof.

It will be appreciated that sensors for use in the monitoring system can be individually incorporated, or can be incorporated as part of a larger system, such as a commercially-available security system, commercially-available health monitoring systems (Daylink Monitor from Alere Medical, Waltham, Mass., USA), or energy monitoring systems, or in combinations thereof.

It is possible to distinguish between the usage of different appliances by their characteristic energy demands. For example, during use, a kettle has a different energy draw profile compared with a microwave oven. By building up a profile of appliances as a library of profiles for a computer program to search against it is possible to map the normal activity of an individual by linking energy usage against the type of appliance being used at a particular time.

Using data from the various sensors, the database of the individual's activity profile around their residence can be built up over a period of time. Preferably, the initial data can be gathered for a time period of from about 1 week to about 12 weeks, more preferably the data can be gathered over a 4 week period, and even more preferably over a 2 week period. This activity profile can be used to define the parameters of “normal” activity and health of that individual, and can be made up of one or more of a number of sensing inputs from the sensing elements.

Examples of aspects of an individual's health that can be monitored include weight, body mass index, waist circumference, bioelectrical impedance analysis (BIA), systolic blood pressure, pulse pressure, and finger-stick blood sampling for biological markers. Typical biological markers tested are selected from the group consisting of glucose, total cholesterol, natriuretic peptides, markers of inflammation (e.g., CD4 and CD8), and combinations of the foregoing. There are several known algorithms that can be used to predict the risk of Cardiovascular disease from these markers. For example, the Framingham Score is a risk assessment tool for estimating 10 year risk of a cardiovascular event. This tool is designed to estimate risk in adults aged 20 years and older who do not have heart disease or diabetes. This risk assessment tool uses age, gender, total and HDL cholesterol, smoker/non-smoker, systolic blood pressure, and assesses use of anti-hypertensive medication. HeartScore® can also be used. The European Society of Cardiology initiated the development of a predictive risk scoring system (SCORE) for predicting and managing the risk of heart attacks and strokes. This system was derived from 12 European cohort studies (n=205,178) containing 3-million person-years of observation and 7,934 fatal cardiovascular events. The evidence-based risk scoring is based upon the country of residence, systolic blood pressure, total cholesterol, smoker/non-smoker and gender.

As previously mentioned, the data transmitted or collected from the sensing elements can be used to establish or set normal behaviour and health parameters for the individual. The data can be collected and either processed within the household or sent to a separate site for processing. Automatic data collection and analysis can be implemented using a computer program (i.e., algorithm) for assessing, preferably in real time, the data transmitted or collected from the sensors. The computer algorithm compares the collected data to threshold values, which are set by the activity profile parameters for the individual, and makes a determination whether the data meets or exceeds those values (depending upon how the algorithm is programmed). Values that are outside the pre-set parameters can be automatically recognized by the program which displays the information or triggers an alarm. The alarm could be issued in a number of ways. For example, an alarm could be a sound that is used to alert the individual or care giver in the residence. The alarm could be sent as a text message to a mobile telephone or as a pre-dialed telephone number to alert the emergency services. The alarm could also be provided in the form of instructions as to what course of action to take by the user. This alarm can be reported to a third party to enable intervention. Intervention may typically entail investigating unusual movement activity within the household which may indicate an intruder or it may indicate an episode of dementia of the individual being monitored. Other interventions could be via a telephone call or an alert to the individual to enquire about the health of the individual. The data can be presented on a web site for the individual to access and for them to monitor their own progress. The data collected and analysed by the computer may also be used by a physician or health care provide to monitor the individual. In this manner, the physician may detect deviations from the normal activity profile and adjust treatment or diagnosis accordingly. This data can be used to directly improve energy efficiency, conserve water usage, and/or improve individual health status.

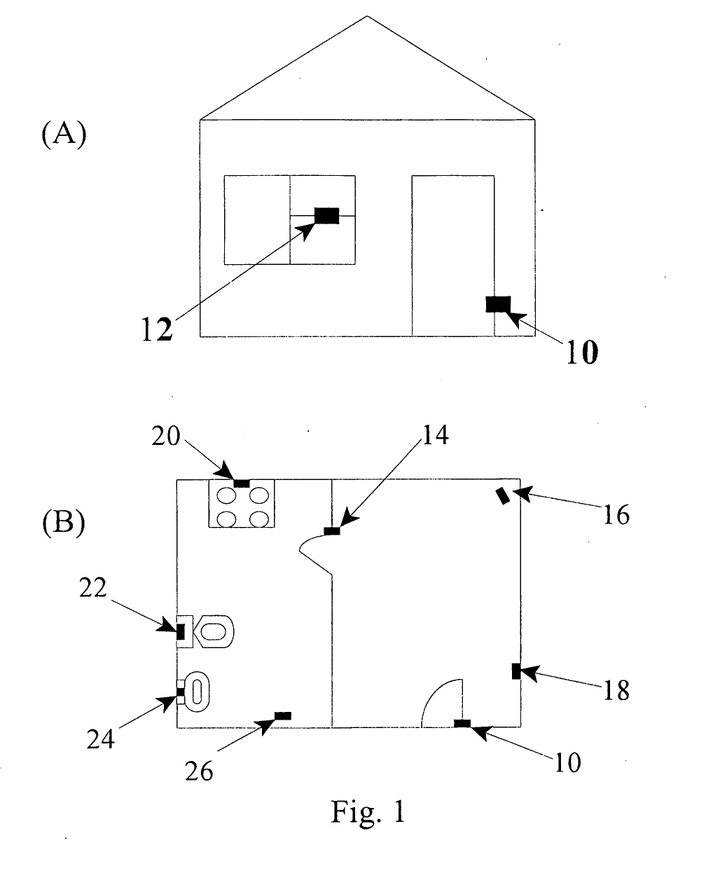

A schematic showing the sensing elements in a typical household is illustrated in FIG. 1(A)-(B). A magnetic door strip 10 is present on an external door. A magnetic window strip 12 is present on a window. A magnetic door strip 14 is present on an internal door. A passive infrared sensor (PIR) 16 is present in the corner of one of the internal rooms. A energy sensing element 18 is present on the power supply entering the household to monitor electricity usage. A sensing element 20 is present on a gas cooker appliance. A sensing element 22 is present on a toilet to monitor water and toilet usage. Another sensing element 24 is present on a tap to monitor usage. A sensing element 26 is present in a remote health monitoring device to measure one or more of the individual's health parameters, as described herein.

In more detail, upon waking, getting out of bed, and moving around their residence, an individual is detected by a PIR sensor 16 at 07:30 hrs. The individual then moves into another room and a magnetic contact sensor 14 detects the door opening between the two rooms at 07:31 hrs. The individual then uses the toilet and upon flushing, a water flow sensor 22 detects water flow at 07:35 hrs. The individual then washes their hands in the sink and an additional water flow sensor 24 detects usage of the tap in the sink at 07:36 hrs. The individual then prepares breakfast and in doing so turns on the gas cooker and a gas sensor 20 detects gas flow at 07:43 hrs. These sensors acquire data that can be automatically transmitted to a computer via general packet radio system (GPRS) technology, or other suitable data transfer method described herein, as each sensor is activated and the computer program logs the acquired data, preferably in real time.

The following day the individual gets up and performs the same activities, but at the following times. The individual wakes, gets out of bed, and is detected by a PIR sensor 16 at 07:38 hrs. The individual then moves into another room and the magnetic door sensor 14 does not detect door opening or closing, as the door was left open the night before. However, the water flow sensor 22 detects water flow at 07:44 hr, and the water flow sensor 24 connected to the sink detects water flow at 07:46. The individual then prepares breakfast and in doing so turns on the gas cooker and a gas sensor 20 detects gas flow at 07:55 hrs. Again, the sensors transmit their data to a computer which logs the acquired data. The foregoing data collection by the computer continues for a predetermined period of time, as described herein, to construct the individual's activity profile and establish the parameters defining “normal” behavior for that individual. Preferably, this profile is automatically developed by the computer program. Alternatively, the profile of “normal” behavior can be set manually by the user or a third party (physician, etc.).

The data from sensors around the residence is preferably automatically collected on a computer programmed with an algorithm for assessing, preferably in real time, the acquired data. The sensors can be of a type as described previously and the data can be sent either wirelessly via Bluetooth, infrared, or Zigbee, or through hard-wired cables to a central hub or to a remote location using GPRS technology currently well known in the art. The remote location could use internet protocol to enable data to be delivered and accessed at a location out with the home. The sensors used in the system can use Smart meter standards for communications. These standards have been set by the Continua Alliance (IEEE 11073) and define communications between personal telehealth devices and computers. The software for enabling this communication between the different sensors and a central hub or processing unit can use Java Card technology.

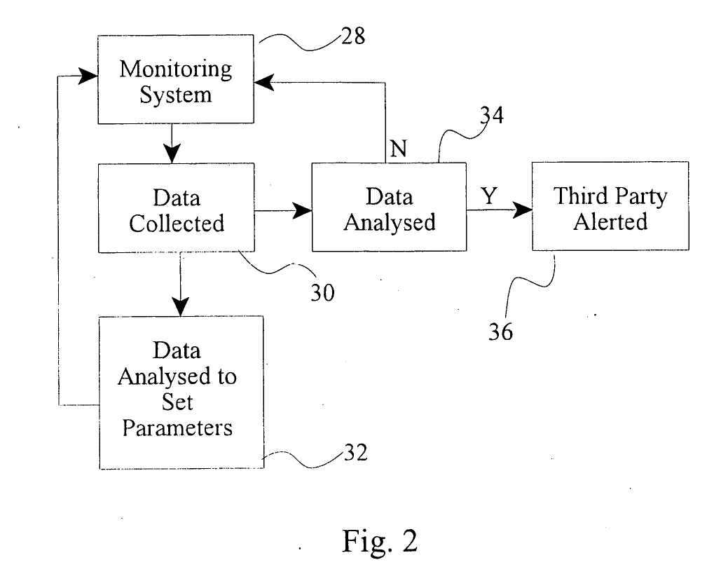

FIG. 2 shows a schematic of how the system is operated. The monitoring system 28 containing the sensing elements (10-26) is set up so that data can be collected 30. The initial data is analysed to set parameters 32 that will be monitored and which define the individual's activity profile. The parameters and threshold values can be automatically set by a computer program. Alternatively, the threshold values and level of tolerance for deviations from the parameters can be manually set. The system 28 is used and data collected 30 and this data is analysed 34, preferably by a computer program, to determine whether the data is outside the set parameters. If the data falls within the defined parameters then the system 28 continues to be used for monitoring. If the data falls outside the defined parameters then an alarm is triggered and a third party is alerted 36. This determination can be automatically performed by a computer, or manually detected, for example by a physician or other healthcare worker.

For example, with reference again to the hypothetical individual and FIG. 1 above, the activity profile for the individual would be based upon a PIR sensor 16 detecting movement between 07:00 and 08:00 hrs, and followed within 15 minutes of detecting movement, a water flow sensor 22 detecting water flow. This would be followed within 5 minutes of the water flow sensor 22 detecting water flow, by a second water flow sensor 24 detecting water flow. After the parameters derived from the activity profile are set, subsequent data is acquired and compared to the set parameters to determine if the data meets or exceeds the threshold values.

In particular, after the parameters are set, the individual gets out of bed upon waking and is detected by the PIR sensor 16 at 07:50 hrs. At 07:55 hrs, the water flow sensor 22 detects water flow and at 07:56 hrs a second water flow sensor 24 detects water flow. These sensors transmit data to a computer as each sensor is triggered and this of data is compared, preferably automatically, to the set parameters (See 34 in FIG. 2). In this example, the set of data collected meets the set parameters and the system continues to monitor (FIGS. 2, 28).

In another embodiment, after the parameters are set, the individual gets out of bed upon waking and is detected by the PIR sensor 16 at 07:20 hrs, and this data is sent to the computer. No other sensors (i.e., 16, 22, 20, or 24) detect activity for 16 minutes. At this point in time the data that has been collected is analysed (see FIGS. 2, 34) and is determined to have deviated from the set parameters (i.e., it meets or exceeds the threshold values). The computer logging the data can be used to compare the acquired data against the set parameters and trigger an alarm or send a signal to alert a third party (i.e., a caregiver) or the individual in the house (FIGS. 2, 36).

It will be appreciated that the set parameters defining normal behavior can consist of more than one rule. As an example, in the above scenario an additional rule could be set such that the water flow sensor 24 should detect water flow within 15 minutes of water flow sensor 22 unless another sensor is activated. These sensing elements can be combined in a number of different ways to monitor an individual's health, as described in more detail below. In the simplest form, monitoring the daily activities of an individual through the pattern of their daily energy usage can be undertaken. The set parameters can also be defined by a combination of probabilities based on the acquired data. For example, an individual may turn on the shower at 08:00 hrs +/−15 minutes with a probability of 95% of the time every day and then turn on a kettle at 09:00 hrs +/−30 minutes with a probability of 75% of the time every day. This may be followed at 10:00 hrs +/−30 minutes with a probability of 50% of turning on a microwave oven. Advantageously, these patterns of energy usage can be used to define an individual's state of health and variations from this behaviour can be used to alert care givers.

As mentioned, the threshold parameters can be manually set, or they can be automatically generated by computer. Suitable algorithms for setting threshold parameters and issuing alerts are known in the art. For example, U.S. Pat. No. 5,609,268, incorporated by reference herein, describes an automatic pill dispensing apparatus, which uses an algorithm to determine the dispensing times of medication and sounds an alarm to instruct the user. In Canadian Patent No. 1239210, incorporated by reference herein, a system for monitoring security guard tours utilizes a pre-defined sequence of events, which are set in a processing algorithm. When deviations from the set algorithm are identified by the central processor an alarm is generated. Likewise, in U.S. Pat. No. 4,857,912, incorporated by reference herein, a multiplicity of sensors are used to detect intrusion into an area. A computing system receives the outputs of the sensors and is programmed to provide an output based on an algorithm. The computing system sums the weighting factors assigned to each sensor in the “on” state, and compares this sum to a reference value and then produces a further output when the sum exceeds that reference. Examples of parameters that can be set in any suitable algorithms known in the art for use in the present invention are described below. It will be appreciated that the parameters and threshold values will vary depending upon the desired use of the system and the specific aspects of the individual's health and/or safety that are of interest to the monitoring party (i.e., the physician, care giver, or individual).

For example, changes in daily weight measured on weighing scales can be measured. The parameter is set as a change in body weight, and the threshold value is met or exceeded (and an alarm is triggered) where the individual's body weight deviates by about 5 lbs or more over about a one week period, or more preferably by about 5 lbs or more over about a 2 day period. Changes in natriuretic peptide levels (for example BNP or NTproBNP) in the blood can be measured. The parameter is set based upon the baseline levels of the individual, and the threshold value is met or exceeded (and an alarm is triggered) where the levels deviate by about 40-200% of the previous baseline measurement, or more preferably by about 40-100% of the previous measurement. Changes in systolic blood pressure can also be measured, where the parameter and threshold value is set based upon the individual's average blood pressure over about 5 or more previous readings, or more preferably over about 3 or more previous readings. A change in systolic blood pressure of greater than about 20% from the initial average reading, or more preferably from about 10-20%, meets or exceeds the threshold value and triggers an alarm. The order of a person's daily activities can be monitored. For example, a pre-defined order of about 5 or more activities or more preferably about 2-5 activities is built up over about a 2 week period and defines the parameters and threshold value for those activities. Deviations or omissions of activity are monitored and a computer running an algorithm provides an output (and/or triggers an alarm) when 2 or more activities are outside of the pre-define order. The timing of expected activities can also be set as a threshold, and an output generated when the timing of activities does not occur within a specified time.

These various parameters can also be combined in a single rule and threshold values can be set based upon a selected combination of activities meeting or exceeding the threshold parameters. For example, the computer may be programmed to recognize when a body weight has increased by 3 lbs from the previous body weight measurement in combination with a 50% decrease in general activity movement around the residence as monitored by the PIR sensors. When the computer makes the determination that both of these threshold values are met or exceeded, it generates an output (and/or triggers an alarm) accordingly.

As previously mentioned, the individual's monitoring profile can be automatically built up through the use of a number of types of sensing elements. These can include, but not be limited to: infrared movement sensors used in home security systems, magnetic door opening and closing sensors, sensors on water usage, an energy sensor that monitors electrical energy draw and then maps this against the type of appliance being used, specific health monitoring sensors that are linked to remote monitoring programmes. The simplest health monitoring sensing elements would be following an individual routine energy usage and identifying variations from set normal parameters. The next preferred combination of sensing elements are monitoring energy usage along with monitoring security sensing elements, like infrared sensors and/or magnetic door opening and closing sensors. Further combinations can be undertaken using a variety of sensing elements. Exemplary combinations of sensors that can be used in the present monitoring system are found in Table 1 below, where “x” indicates that the sensing element is being used in the combination.

TABLE 1

| Sensing Element | Combinations | | | | | | | | | | | | | | | | | | | | | | |
|---|---|---|---|---|---|---|---|---|---|---|---|---|---|---|---|---|---|---|---|---|---|---|---|
| PIR | | x | x | x | | | | | x | x | | x | | x | | x | x | x | x | x | x | x | x |
| Magnetic door strip | | | | x | | | | | | | x | x | | | x | x | x | | | | | | x |
| Electrical appliance or | x | | x | x | x | x | x | x | | x | x | x | x | x | x | x | x | x | | x | x | x | x |
| usage (energy sensor) | | | | | | | | | | | | | | | | | | | | | | | |
| Water usage | | | | | | | | | | | | | x | x | x | x | x | | | | x | x | x |
| Gas usage | | | | | | | | | | | | | | | | | x | | | | | x | x |
| Weight scales | | | | | x | x | x | x | x | x | x | x | x | x | x | x | x | x | x | x | x | x | x |
| BIA | | | | | | | x | x | | | | | | | | | | x | | | | x | x |
| Blood pressure | | | | | | x | x | x | x | x | x | x | x | x | x | x | x | x | x | x | x | x | x |
| Finger-stick biological | | | | | | | | x | | | | | | | | | | | x | x | x | x | x |
| marker | | | | | | | | | | | | | | | | | | | | | | | |

I claim:

1. A method of remotely monitoring the security of a property or the health of an individual using a remote monitoring system, said system comprising:
at least one of a first sensing element that acquires data regarding movement of said individual; and
at least one of a second sensing element that acquires data regarding health of said individual.

2. The method of claim 1 wherein said first sensing element is selected from the group consisting of a passive infrared sensor, a magnetic door strip sensor, a water flow sensor, a gas flow sensor, an energy sensor, and combinations thereof.

3. The method of claim 1, wherein said second sensing element is selected from the group consisting of an energy sensor, a water flow sensor, a gas flow sensor, a weight scale, a bioelectrical impedance sensor, a blood pressure sensor, a pulse pressure sensor, a finger-stick blood test, a blood glucose concentration test, a test for measuring natriuretic peptides, a test for measuring viral load, a human body fluid test, and combinations thereof, said human body fluid test being capable of detecting cholesterol, HDL, LDL, or combinations thereof.

4. The method of claim 1, said system further comprising at least two of said second sensing elements, wherein said first sensing element is a passive infrared sensor and said second sensing elements are individually selected from the group consisting of a weight scale, a bioelectrical impedance sensor, a blood pressure sensor, a pulse pressure sensor, a finger-stick blood test, a blood glucose concentration test, a test for measuring natriuretic peptides, a test for measuring viral load, a human body fluid test, and combinations thereof, said human body fluid test being capable of detecting cholesterol, HDL, LDL, or combinations thereof.

5. The method of claim 1, said system further comprising at least two of said first sensing elements, and at least two of said second sensing elements, wherein said first sensing elements are individually selected from the group consisting of a passive infrared sensor, an energy sensor, and combinations thereof, and said second sensing elements are individually selected from the group consisting of a weight scale, a bioelectrical impedance sensor, a blood pressure sensor, a pulse pressure sensor, a finger-stick blood test, and combinations thereof.

6. A remote monitoring system for monitoring the activity of an individual comprising:
at least one of a first sensing element that acquires data regarding the movement of said individual; and
at least one of a second sensing element that acquires data regarding the health of said individual.

7. The system of claim 6, wherein said first sensing element is passive infrared sensor, and said second sensing element is selected from the group consisting of weight scales, a blood pressure sensor, a pulse pressure sensor, and combinations thereof.

8. The system of claim 6, wherein said first sensing element is selected from the group consisting of a passive infrared sensor, an energy sensor, and combinations thereof, and said second sensing element is selected from the group consisting of a weight scale, a blood pressure sensor, a pulse pressure sensor, and combinations thereof.

9. The system of 6, wherein said first sensing element is selected from the group consisting of a passive infrared sensor, an energy sensor, and combinations thereof, and said second sensing element is selected from the group consisting of a weight scale, a bioelectrical impedance sensor, a blood pressure sensor, a pulse pressure sensor, and combinations thereof.

10. The system of claim 6, wherein said first sensing element is selected from the group consisting of a passive infrared sensor, an energy sensor, and combinations thereof, and said second sensing element is selected from the group consisting of a weight scale, a finger-stick blood test, a blood pressure sensor, a pulse pressure sensor, and combinations thereof.

11. The system of claim 6, further comprising a computer for collecting said data from said first and second sensing elements.

12. A method of remotely monitoring the activity of an individual comprising:
collecting a first set of data from a sensing element, said sensing element being selected from group consisting of a passive infrared sensor, a magnetic door strip sensor, an energy sensor, a water flow sensor, a gas flow sensor, a weight scale, a bioelectrical impedance sensor, a blood pressure sensor, a pulse pressure sensor, a finger-stick blood test, a blood glucose concentration test, a test for measuring natriuretic peptides, a test for measuring viral load, a human body fluid test, and combinations thereof, said human body fluid test being capable of detecting cholesterol, HDL, LDL, or combinations thereof;
analyzing said first set of data to develop an activity profile for said individual, said activity profile defining parameters of normal activity levels of said individual;
collecting a second set of data from said sensing element; and
comparing said second set of data to said parameters.

13. The method of claim 12, further comprising:
triggering an alarm if said second set of data deviates from said parameters.

14. The method of claim 13, wherein the determination of whether said second set of data deviates from said parameters is automatically performed by a computer program.

15. The method of claim 12, further comprising:
alerting said individual if said second set of data deviates from said parameters.

16. The method of claim 12, wherein said first set of data is collected over a time period of from about 1 week to about 12 weeks.

17. The method of claim 12, wherein said analyzing of said first set of data is performed by a computer program.

18. The method of claim 17, wherein said activity profile is automatically developed by said computer program.

19. The method of claim 12, wherein said first and second sets of data are automatically collected using a computer.

20. The method of claim 12, wherein said comparing said second set of data to said parameters is automatically performed by a computer program.
